(19) United States
(12) Patent Application Publication
(10) Pub. No.: US 2025/0381067 A1
(43) Pub. Date: Dec. 18, 2025

(54) APPARATUS FOR DELIVERING OCULAR IMPLANTS INTO AN ANTERIOR CHAMBER OF THE EYE

(71) Applicant: Alcon Inc., Fribourg (CH)

(72) Inventors: Andrew T. SCHIEBER, St. Louis Park, MN (US); Iqbal K. AHMED, Mississauga (CA)

(73) Assignee: Alcon Inc., Fribourg (CH)

(21) Appl. No.: 19/261,135

(22) Filed: Jul. 7, 2025

Related U.S. Application Data
(63) Continuation of application No. 18/334,256, filed on Jun. 13, 2023, now Pat. No. 12,376,988.
Application No. 18/334,256 is a continuation of application No. 16/805,217, filed on Feb. 28, 2020, now Pat. No. 11,712,369.
Application No. 16/805,217 is a continuation of application No. 14/440,610, filed on May 5, 2015, now Pat. No. 10,617,558.
(60) Provisional application No. 61/730,895, filed on Nov. 28, 2012.

(51) Int. Cl. A61F 9/00; A61F 9/007

(52) U.S. Cl. CPC A61F 9/0017; A61F 9/00781

(57) ABSTRACT

An ocular implant adapted to be disposed within Schlemm's canal of a human eye with a body extending along a curved longitudinal central axis in a curvature plane, a first strut on one side of the implant and a second strut on an opposite side of the implant, the circumferential extent of the first strut with respect to the plane of curvature being greater than the circumferential extent of the second strut with respect to the plane of curvature. The invention also includes methods of using the implant.

### CROSS REFERENCE TO RELATED APPLICATIONS

This application is a continuation U.S. patent application Ser. No. 18/334,256, filed Jun. 13, 2023, which is a continuation U.S. patent application Ser. No. 16/805,217, filed Feb. 28, 2020, now U.S. Pat. No. 11,712,369, which is a continuation of U.S. patent application Ser. No. 14/440,610, filed May 5, 2015, now U.S. Pat. No. 10,617,558, which application is the national stage of International Application No. PCT/US2013/072001, filed Nov. 26, 2013, which application claims the benefit of U.S. Provisional Application No. 61/730,895, filed Nov. 28, 2012, the disclosures of which are incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure relates generally to devices that are implanted within the eye. More particularly, the present disclosure relates to systems, devices and methods for delivering ocular implants into the eye.

### BACKGROUND

According to a draft report by The National Eye Institute (NEI) at The United States National Institutes of Health (NIH), glaucoma is now the leading cause of irreversible blindness worldwide and the second leading cause of blindness, behind cataract, in the world. Thus, the NEI draft report concludes, “it is critical that significant emphasis and resources continue to be devoted to determining the pathophysiology and management of this disease.” Glaucoma researchers have found a strong correlation between high intraocular pressure and glaucoma. For this reason, eye care professionals routinely screen patients for glaucoma by measuring intraocular pressure using a device known as a tonometer. Many modem tonometers make this measurement by blowing a sudden puff of air against the outer surface of the eye.

The eye can be conceptualized as a ball filled with fluid. There are two types of fluid inside the eye. The cavity behind the lens is filled with a viscous fluid known as vitreous humor. The cavities in front of the lens are filled with a fluid know as aqueous humor. Whenever a person views an object, he or she is viewing that object through both the vitreous humor and the aqueous humor.

Whenever a person views an object, he or she is also viewing that object through the cornea and the lens of the eye. In order to be transparent, the cornea and the lens can include no blood vessels. Accordingly, no blood flows through the cornea and the lens to provide nutrition to these tissues and to remove wastes from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the anterior chamber of the eye through the trabecular meshwork and into Schlemm's canal as new aqueous humor is secreted by the epithelial cells of the ciliary body. This excess aqueous humor enters the venous blood stream from Schlemm's canal and is carried along with the venous blood leaving the eye.

When the natural drainage mechanisms of the eye stop functioning properly, the pressure inside the eye begins to rise. Researchers have theorized prolonged exposure to high intraocular pressure causes damage to the optic nerve that transmits sensory information from the eye to the brain. This damage to the optic nerve results in loss of peripheral vision. As glaucoma progresses, more and more of the visual field is lost until the patient is completely blind.

In addition to drug treatments, a variety of surgical treatments for glaucoma have been performed. For example, shunts were implanted to direct aqueous humor from the anterior chamber to the extraocular vein (Lee and Scheppens, “Aqueous-venous shunt and intraocular pressure,” Investigative Ophthalmology (February 1966)). Other early glaucoma treatment implants led from the anterior chamber to a sub-conjunctival bleb (e.g., U.S. Pat. Nos. 4,968,296 and 5,180,362). Still others were shunts leading from the anterior chamber to a point just inside Schlemm's canal (Spiegel et al., “Schlemm's canal implant: a new method to lower intraocular pressure in patients with POAG?” Ophthalmic Surgery and Lasers (June 1999); U.S. Pat. Nos. 6,450,984; 6,450,984).

### SUMMARY OF THE DISCLOSURE

A cannula for delivering an ocular implant into Schlemm's canal of an eye is provided, comprising a rigid curved tube adapted to extend through an anterior chamber of the eye to achieve tangential entry into Schlemm's canal, a trough portion formed by an opening extending along a distal portion of the rigid curved tube, and an asymmetric tip disposed at a distal end of the trough portion, the asymmetric tip being located at an intersection between an upper camming surface and a lower camming surface, the upper camming surface being configured to contact scleral tissue of the eye to guide the trough portion into Schlemm's canal, the lower camming surface being configured to contact a scleral spur of the eye to guide the trough portion into Schlemm's canal.

In some embodiments, the asymmetric tip is configured to not pierce the scleral tissue. In other embodiments, the asymmetric tip is configured to pierce the trabecular meshwork. In some embodiments, the asymmetric tip is formed by the upper camming surface being shorter than the lower camming surface.

In one embodiment, the rigid curved tube and the trough portion define a path for directing the ocular implant from a location outside of the eye to a location within Schlemm's canal of the eye.

In some embodiments, the asymmetric tip is sufficiently blunt to slide along an outer wall of Schlemm's canal without cutting the scleral tissue underlying the outer wall of Schlemm's canal.

In one embodiment, the asymmetric tip has an asymmetric V-shape.

In some embodiments, the cannula is shaped and dimensioned so that at least part some of the trough portion can be advanced into Schlemm's canal while a first portion of the rigid curved tube is disposed inside the anterior chamber and a second portion of the rigid curved tube is extended through an incision in the eye to a location outside of the eye.

An ocular implant and delivery system is also provided, comprising a rigid curved cannula adapted to extend through an anterior chamber of an eye to achieve tangential entry into Schlemm's canal of the eye, a trough portion formed by an opening extending along a distal portion of the rigid curved cannula, an ocular implant configured to be carried inside the rigid curved cannula and advanced distally through the rigid curved cannula and along the trough portion into Schlemm's canal, and an asymmetric tip 9 disposed at a distal end of the trough portion, the asymmetric tip being located at an intersection between an upper camming surface and a lower camming surface, the upper camming surface being configured to contact scleral tissue of the eye to guide the trough portion into Schlemm's canal, the lower camming surface being configured to contact a scleral spur of the eye to guide the trough portion into Schlemm's canal.

In some embodiments, the asymmetric tip is configured to not pierce the scleral tissue. In other embodiments, the asymmetric tip is configured to pierce the trabecular meshwork. In some embodiments, the asymmetric tip is formed by the upper camming surface being shorter than the lower camming surface.

In one embodiment, the rigid curved tube and the trough portion define a path for directing the ocular implant from a location outside of the eye to a location within Schlemm's canal of the eye.

In some embodiments, the asymmetric tip is sufficiently blunt to slide along an outer wall of Schlemm's canal without cutting the scleral tissue underlying the outer wall of Schlemm's canal.

In one embodiment, the asymmetric tip has an asymmetric V-shape.

In some embodiments, the cannula is shaped and dimensioned so that at least part some of the trough portion can be advanced into Schlemm's canal while a first portion of the rigid curved tube is disposed inside the anterior chamber and a second portion of the rigid curved tube is extended through an incision in the eye to a location outside of the eye.

In some embodiments, the rigid curved cannula and the trough portion define a path for directing the ocular implant from a location outside of the eye to a location within Schlemm's canal of the eye.

In another embodiment, the asymmetric tip is sufficiently blunt to slide along an outer wall of Schlemm's canal without cutting the scleral tissue underlying the outer wall of Schlemm's canal.

In some embodiments, the asymmetric tip has an asymmetric V-shape.

In another embodiment, the rigid curved cannula is shaped and dimensioned so that at least part some of the trough portion can be advanced into Schlemm's canal while a first portion of the rigid curved cannula is disposed inside the anterior chamber and a second portion of the rigid curved cannula is extended through an incision in the eye to a location outside of the eye.

A cannula for delivering an ocular implant into Schlemm's canal of an eye is also provided, comprising a rigid body having a distal curved portion adapted to gain tangential entry into Schlemm's canal, a lumen extending from a proximal end of the body through at least part of the distal curved portion, the lumen being adapted to contain the ocular implant, a trough formed in the distal curved portion, the trough being defined by an opening along the body that provides access to a concave inner surface, and a distal tip at a distal end of the trough, the distal tip being in a position offset from a central axis of the trough.

In some embodiments, the distal tip is formed at an intersection between an upper camming surface and a lower camming surface. In one embodiment, the upper camming surface is smaller than the lower camming surface.

In some embodiments, the distal tip is sufficiently blunt to slide along an outer wall of Schlemm's canal without cutting scleral tissue underlying the outer wall of Schlemm's canal.

A method of inserting an ocular implant into Schlemm's canal of an eye is provided, the method comprising inserting a curved cannula having a distal trough portion through an anterior chamber of the eye to gain tangential entry of the trough portion into Schlemm's canal, allowing an upper camming surface of a distal tip of the distal trough portion to contact scleral tissue of the eye to guide the distal trough portion into Schlemm's canal, allowing a lower camming surface of the distal tip of the distal trough portion to contact a scleral spur of the eye to guide the distal trough portion into Schlemm's canal, and advancing an ocular implant through the curved cannula and along the distal trough portion into Schlemm's canal.

In some embodiments of the cannulas described herein, a diameter of the rigid curved tube is larger than a width of Schlemm's canal. In one embodiment, the diameter of the rigid curved tube is approximately 400-500 microns. In another embodiment, the diameter of the rigid curved tube is approximately 350-550 microns.

### BRIEF DESCRIPTION OF THE DRAWINGS

FIG. 1 is a stylized representation of a medical procedure in accordance with this detailed description.

FIG. 2 is an enlarged perspective view further illustrating the delivery system and the eye shown in the previous figure.

FIG. 3A is a perspective view further illustrating the eye and cannula shown in the previous figure.

FIG. 3B is a section view further illustrating the eye shown in FIG. 3A.

FIG. 3C is perspective view further illustrating the anatomy of the eye shown in FIG. 3B.

FIG. 3D is a perspective view showing a portion of eye shown in FIG. 3C.

FIG. 3E is an additional perspective view showing the ocular implant and the cannula shown in FIG. 3D.

FIG. 4 is a photographic image showing a histology slide HS. Histology slide HS of FIG. 4 was created by sectioning and staining tissue from a cadaveric eye. An ocular implant was implanted in Schlemm's canal of the cadaveric eye prior to sectioning.

FIG. 5A is a stylized line drawing illustrating histology slide HS shown in the previous figure.

FIG. 5B is a simplified cross-sectional view illustrating the eye from which the histology sample was taken.

FIG. 6 is a stylized perspective view illustrating the anatomy of an eye.

FIG. 7 is a stylized perspective view depicting the surface that defines the anterior chamber of the eye shown in FIG. 6.

FIG. 8 is a stylized perspective view further illustrating Schlemm's canal SC and iris 30 shown in FIG. 6.

FIGS. 9A-9C are plan views of the surface that defines anterior chamber of the eye shown in FIG. 6.

FIG. 10 is an enlarged side view showing a cannula extending into anterior chamber defined by an inner surface of a dome shaped wall.

FIGS. 11A-11C are plan views of a cannula created using multiview projection.

FIG. 11D is an axial view further illustrating the cannula shown in FIG. 11A.

FIGS. 12A-12D are lateral cross-sectional views of the tip portion of a cannula.

FIG. 12E is a lateral cross-sectional view of a trough portion of the cannula.

FIG. 12F is a plan view of the cannula including a plurality of section lines.

FIGS. 13A-13D form a sequence of stylized section views illustrating the insertion of the tip portion of a cannula into Schlemm's canal located in the anterior chamber of an eye.

FIGS. 13E-13H form a sequence stylized side plan views further illustrating the insertion of the tip portion into Schlemm's canal.

FIG. 14 is an abstract graphical representation further illustrating the insertion of the tip portion of a cannula into Schlemm's canal.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

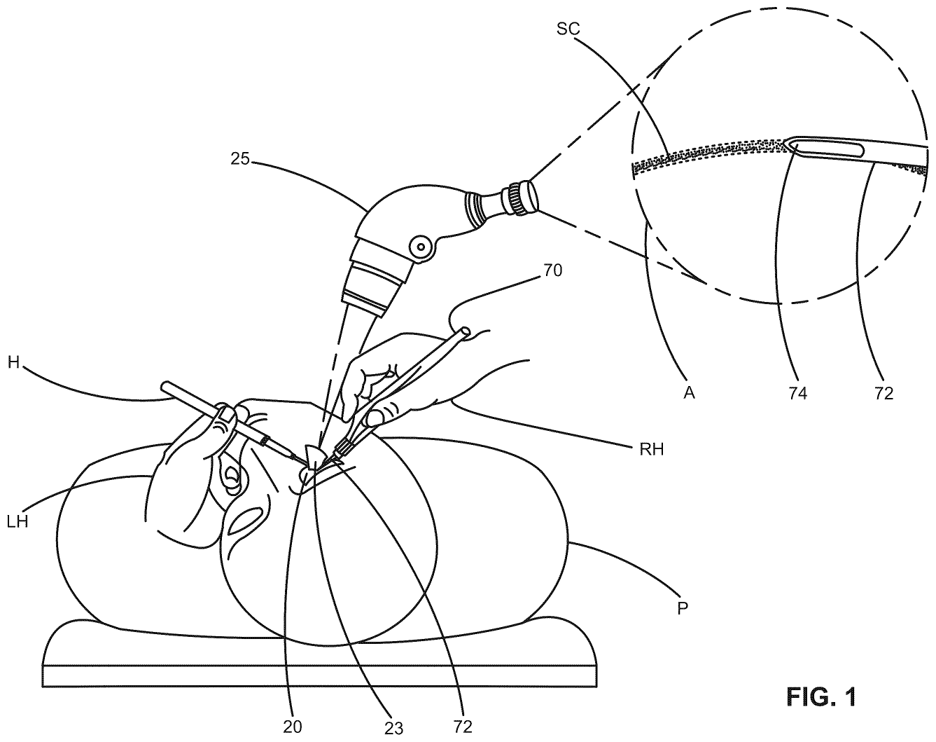

FIG. 1 is a stylized representation of a medical procedure in accordance with this detailed description. In the procedure of FIG. 1, a physician is treating an eye 20 of a patient P. The physician is holding a hand piece of a delivery system 70 in his or her right hand RH. The physician's left hand LH is holding the handle H of a gonio lens 23 in the procedure of FIG. 1. Some physicians may prefer holding the delivery system hand piece in the right hand and the gonio lens handle in the left hand.

During the procedure illustrated in FIG. 1, the physician may view the interior of the anterior chamber using gonio lens 23 and a microscope 25. Detail A of FIG. 1 is a stylized simulation of the image viewed by the physician. A distal portion of a cannula 72 is visible in Detail A. A shadow-like line indicates the location of Schlemm's canal SC, which is a tube-like structure that encircling the iris and lying under various tissue (e.g., the trabecular meshwork) that surround the anterior chamber. A distal opening 74 of cannula 72 is positioned near Schlemm's canal SC of eye 20.

Methods in accordance with this detailed description may include the step of advancing the distal end of cannula 72 through the cornea of eye 20 so that a distal portion of cannula 72 is disposed in the anterior chamber of the eye. Cannula 72 may then be used to access Schlemm's canal of the eye, for example, by piercing the wall of Schlemm's canal with the distal end of cannula 72. Distal opening 74 of cannula 72 may be placed in fluid communication with a lumen defined by Schlemm's canal. An ocular implant carried by the cannula may be advanced out of distal opening 74 and into Schlemm's canal. Insertion of the ocular implant into Schlemm's canal may facilitate the flow of aqueous humor out of the anterior chamber of the eye. Examples of ocular implants that may be delivered through the cannula of this invention may be found, e.g., in U.S. Pat. Nos. 7,740,604; 8,267,882; 8,425,449; US Patent Publ. No. 2009/0082860 (now U.S. Pat. No. 8,734,377); and US Patent Publ. No. 2009/0082862.

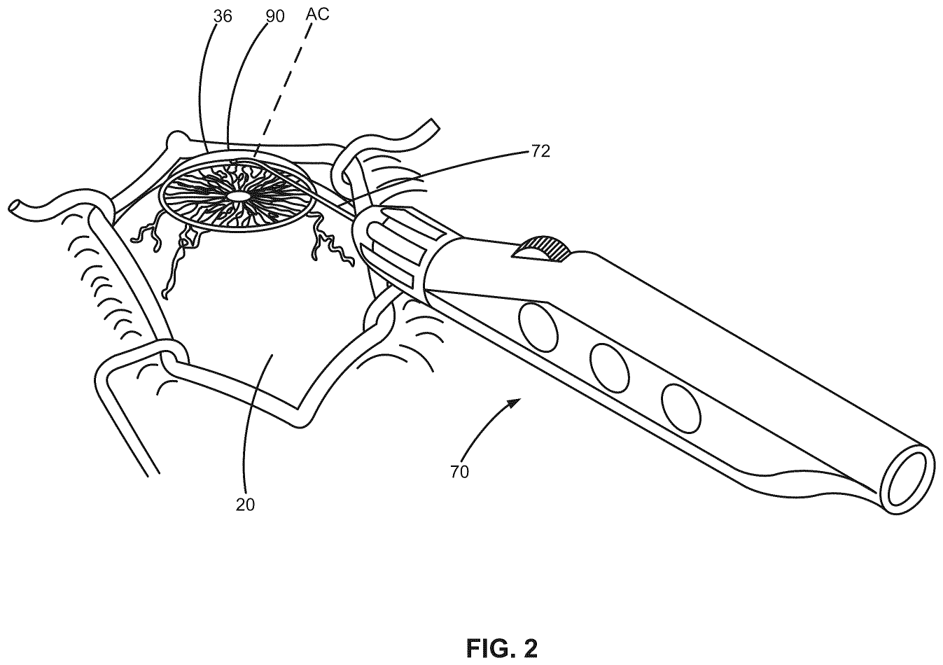

FIG. 2 is an enlarged perspective view further illustrating delivery system 70 and eye 20 shown in the previous figure. In FIG. 2, cannula 72 of delivery system 70 is shown being advanced and extending through a dome-shaped wall 90 of eye 20. Dome shaped wall 90 includes the cornea 36 of eye 20 and scleral tissue that meets the cornea at a limbus of the eye. A distal portion of cannula 72 is disposed inside the anterior chamber AC defined by dome-shaped wall 90. In the embodiment of FIG. 2, cannula 72 is sized and configured so that a distal opening of cannula 72 can be placed in fluid communication with Schlemm's canal while a proximal portion of cannula 72 is extending through an incision in cornea 36.

In the embodiment of FIG. 2, an ocular implant (not shown) is disposed in a lumen or passageway within cannula 72. Delivery system 70 includes a mechanism that is capable of advancing and retracting the ocular implant along the length of cannula 72. Suitable delivery systems are described in more detail in, e.g., U.S. Pat. Nos. 8,512,404; 8,337,509; US Patent Publ. No. 2011/0009874 (now U.S. Pat. No. 9,693,899); and US Patent Publ. No. 2013/0158462 (now U.S. Pat. No. 8,663,150). The ocular implant may be placed in Schlemm's canal of eye 20 by advancing the ocular implant through the distal opening of cannula 72 while the distal opening is in fluid communication with Schlemm's canal.

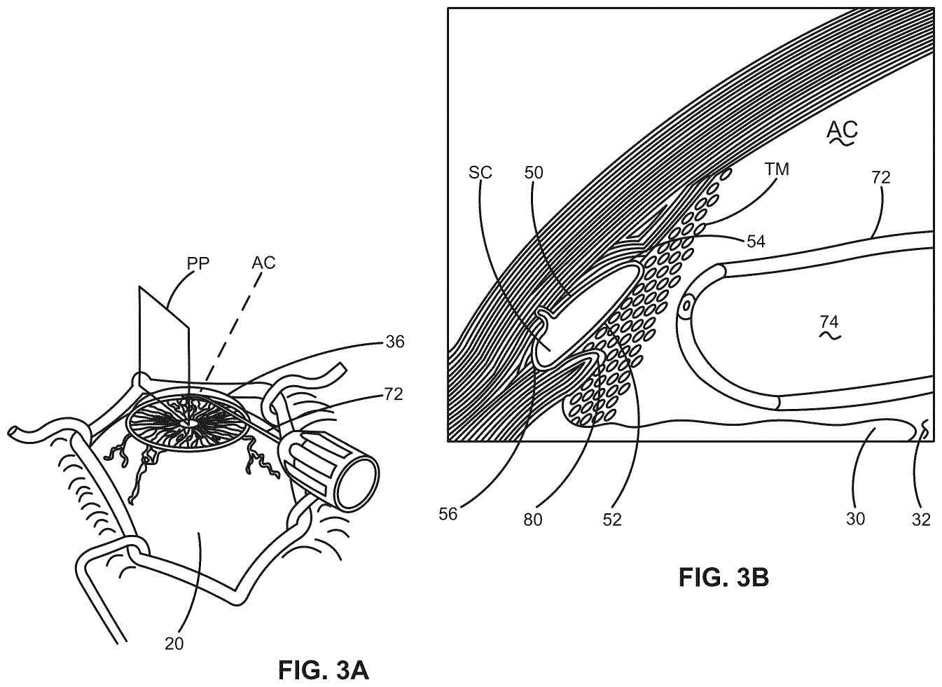

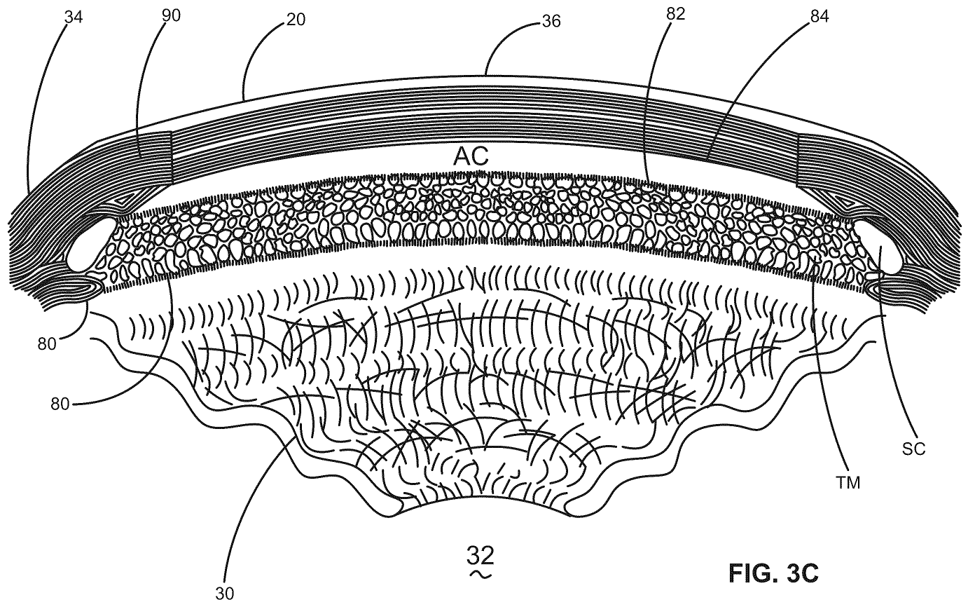

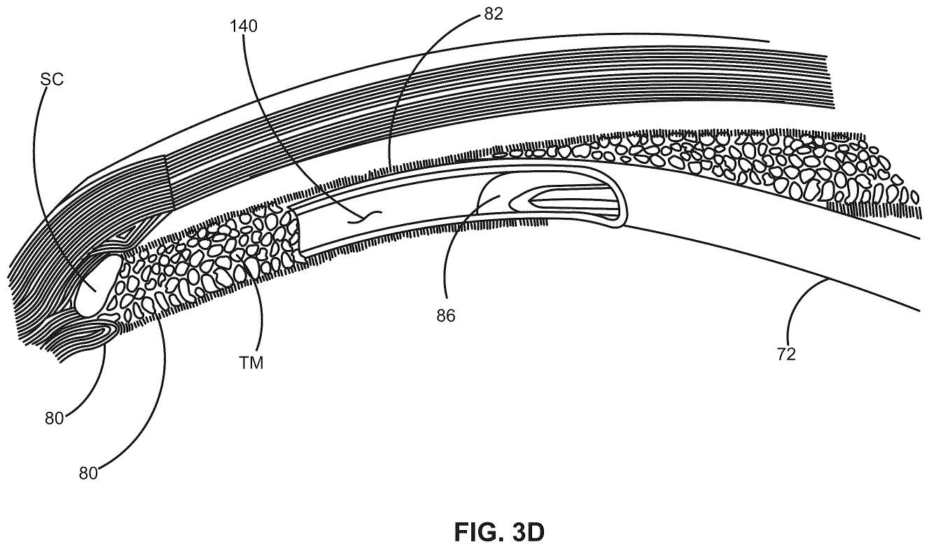

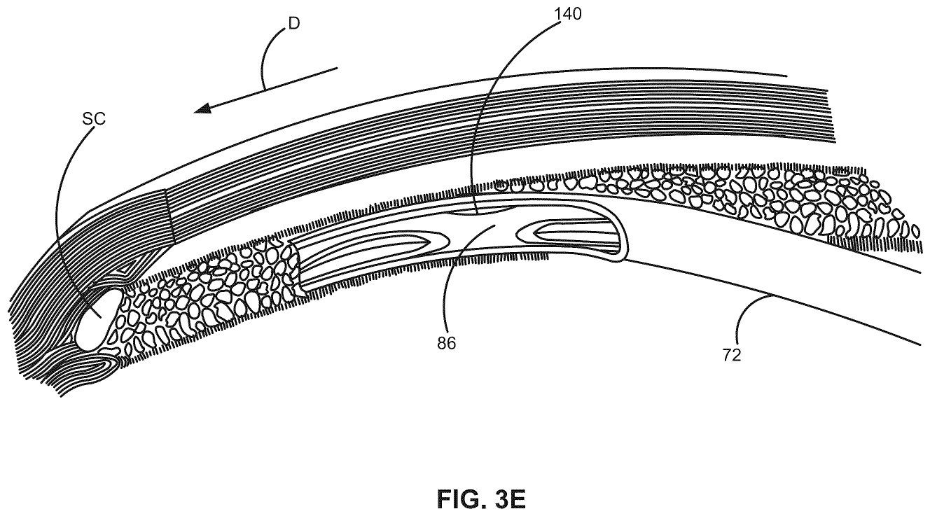

FIG. 3A is a perspective view further illustrating eye 20 shown in the previous figure. In FIG. 3A, cannula 72 is shown extending through a cornea 36 of eye 20. In FIG. 3B, a distal opening 74 of cannula 72 is shown disposed inside an anterior chamber AC of eye 20. In FIG. 3A, a cutting plane PP is shown extending across eye 20. FIG. 3B is a stylized cross-sectional view taken along cutting plane PP shown in FIG. 3A. The cutting plane of FIG. 3A extends laterally across Schlemm's canal SC and the trabecular 11 meshwork TM of the eye.

Eye 20 includes an iris 30 that defines a pupil 32 of the eye. Schlemm's canal SC forms a ring around iris 30 with pupil 32 disposed in the center of that ring. Schlemm's canal SC has a first major side 50, a second major side 52, a first minor side 54, and a second minor side 56. First major side 50 is on the outside of the ring formed by Schlemm's canal SC and second major side 52 is on the inside of the ring formed by Schlemm's canal SC. Accordingly, first major side 50 may be referred to as an outer major side of Schlemm's canal SC and second major side 52 may be referred to as an inner major side of Schlemm's canal SC. With particular reference to FIG. 3B, it will be appreciated that first major side 50 is further from pupil 32 than second major side 52. In the schematic view shown in FIG. 3A, first major side 50 is an outer major side of Schlemm's canal SC and second major side 52 is an inner major side of Schlemm's canal SC. A scleral spur 80 extends around minor side 56 toward the trabecular meshwork TM.

FIG. 3C is perspective view further illustrating the anatomy of eye 20 shown in FIG. 3B. Eye 20 includes a dome-shaped wall 90 that defines and encloses the anterior chamber AC. Dome-shaped wall 90 comprises a cornea 36 and scleral tissue 34. The scleral tissue 34 meets the cornea 36 at a limbus of eye 20. Dome-shaped wall 90 includes a scleral spur 80 that encircles anterior chamber AC. Schlemm's canal SC resides in a shallow depression in the scleral tissue located near scleral spur 80. The trabecular meshwork TM is fixed to scleral spur 80 and extends over Schlemm's canal. Together, Schlemm's canal SC, trabecular meshwork TM, and scleral spur 80 encircle anterior chamber AC along dome-shaped wall 90. Iris 30 of eye 20 is disposed inside the anterior chamber AC. Iris 30 defines a pupil 32. Schwalbe's line 82 is disposed at the end of Descemet's membrane 84. Descemet's membrane 84 is one of the inner-most layers of cornea 36. Descemet's membrane extends across cornea 36 toward Schlemm's canal SC and terminates near the upper edge of Schlemm's canal SC.

FIG. 3D is a perspective view showing a portion of eye shown in the previous figure. In FIG. 3D, the tip portion of a cannula 72 can be seen extending into trabecular meshwork TM. In some useful embodiments, cannula 72 can be curved to achieve 9 substantially tangential entry into Schlemm's canal SC. Also in the embodiment of FIG. 3D, a curved distal portion of cannula 72 is dimensioned to be disposed within the anterior chamber of the eye. In FIG. 3D, an ocular implant 86 can be seen extending from a lumen in cannula 72 into a trough 140 defined by cannula 72. Ocular implant 86 can be advanced through a distal opening of cannula 72 along the trough 140 and into Schlemm's canal SC. Scleral spur 80 and Schwalbe's line 82 are also visible in FIG. 3D.

FIG. 3E is an additional perspective view showing ocular implant 86 and cannula 72 shown in the previous figure. By comparing FIG. 3E with the previous figure, it will be appreciated that ocular implant 86 has been advanced in a distal direction D while cannula 72 has remained stationary so the distal end of ocular implant 86 is disposed inside Schlemm's canal SC and the remainder of the implant is disposed in trough 140 and inside the lumen of the cannula. Trough 140 opens into an elongate opening extending through the side wall of cannula 72. In the embodiment of FIG. 3E, the elongate opening defined by the cannula provides direct visualization of the ocular implant as it is advanced into Schlemm's canal. A configuration allowing direct visualization of the ocular implant has a number of clinical advantages. During a medical procedure, it is often difficult to monitor the progress of the implant by viewing the implant through the trabecular meshwork. For example, blood reflux may push blood into Schlemm's canal obstructing a physician's view the portion of the implant that has entered Schlemm's canal. With reference to FIG. 3E, ocular implant 86 tracks along trough 140 as it is advanced distally along cannula 72 into Schlemm's canal. The trough opening allows the physician to monitor the progress of the implant by viewing the implant structures as they advance through the trough prior to entering Schlemm's canal. The trough opening also allows the physician to identify the position of the proximal end of the ocular implant with respect to the incision made by the cannula to access Schlemm's canal.

The ocular implants referenced above are intended to reside partially or wholly within Schlemm's canal. One function of the cannula is to deliver a leading edge of the ocular implant into Schlemm's canal so that the ocular implant can be advanced circumferentially into Schlemm's canal. The cannula of this invention provides features to help the user guide the distal end of the cannula into Schlemm's canal. These cannula features take advantage of the shapes and properties of the various tissue structures of and around Schlemm's canal to achieve this goal.

When inserting a cannula through the anterior chamber and the trabecular meshwork into Schlemm's canal under gonio lens visualization, the physician may use anatomical landmarks to guide the cannula placement and advancement. One convenient landmark is scleral spur 80 which has the appearance of a white line encircling the anterior chamber AC. Another convenient landmark is a pigment line centered on Schlemm's canal SC. An additional convenient landmark is Schwalbe's line 82.

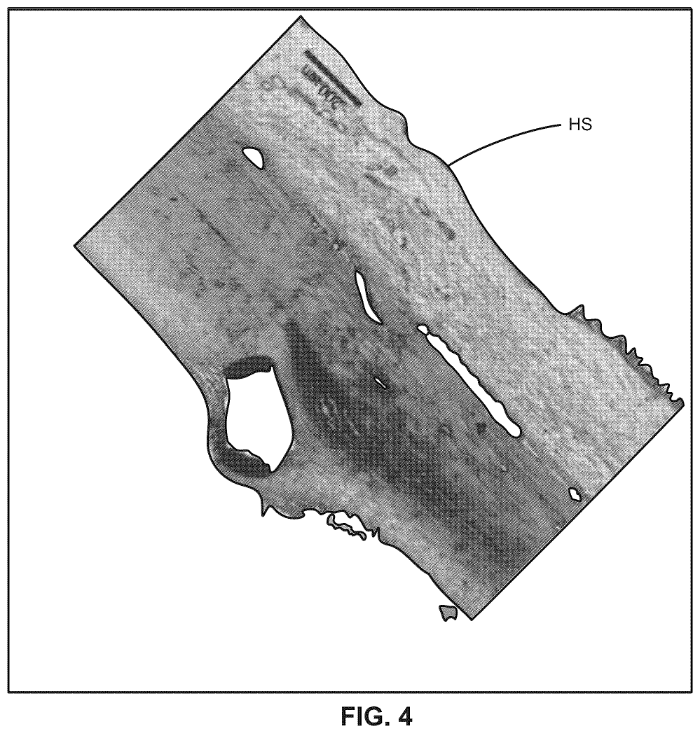

An ocular implant residing in Schlemm's canal of a cadaveric eye can be seen in FIG. 4. FIG. 4 is a photographic image showing a histology slide HS. Histology slide HS of FIG. 4 was created by implanting the ocular implant into Schlemm's canal of the eye, then sectioning and staining a portion of the eye. The photograph of FIG. 4 was created while examining the section of tissue using a light microscope.

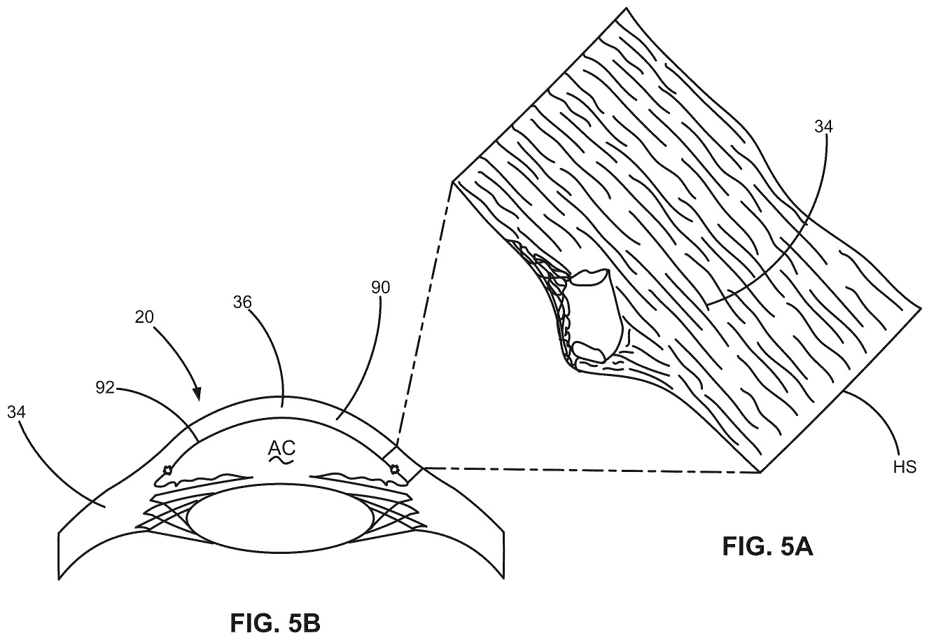

FIG. 5A is a stylized line drawing illustrating histology slide HS shown in the previous figure. FIG. 5B is a simplified cross-sectional view illustrating the eye from which the histology sample was taken. FIG. 5A and FIG. 5B are presented on a single page to illustrate the location of the histology sample relative to other portions of the eye 20. Eye 20 includes a dome-shaped wall 90 having a surface 92 defining an anterior chamber AC. Dome-shaped wall 90 of eye 20 comprises a cornea 36 and scleral tissue 34. The scleral tissue 34 meets the cornea 36 at a limbus of the eye. In FIG. 5B, surface 92 is shown having a generally hemispherical shape.

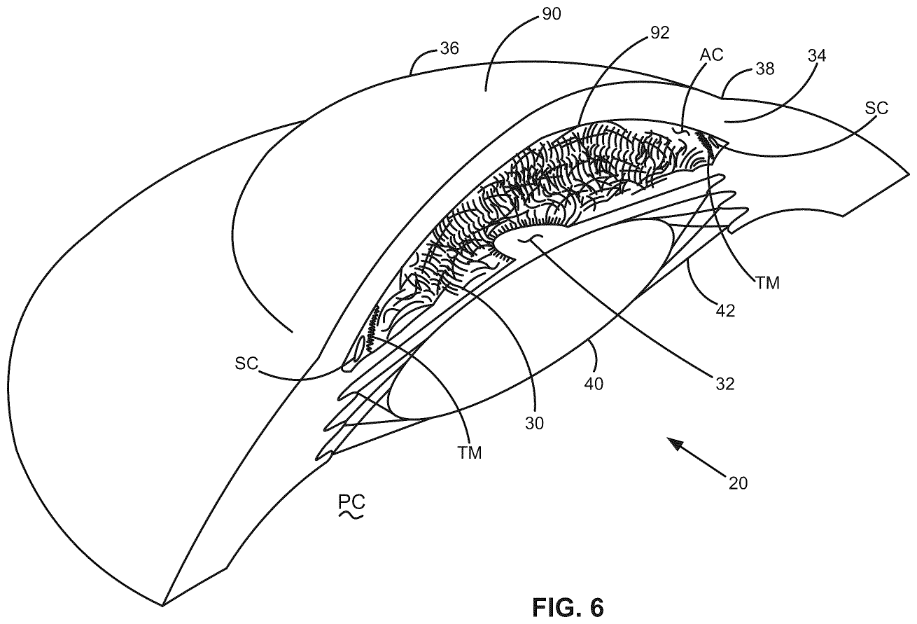

FIG. 6 is a stylized perspective view illustrating a portion of eye 20 discussed above. Eye 20 includes an iris 30 defining a pupil 32. In FIG. 6, eye 20 is illustrated in a cross-sectional view created by a cutting plane passing through the center of pupil 32. Eye 20 includes a dome-shaped wall 90 having a surface 92 defining an anterior chamber AC. In FIG. 6, surface 92 is shown having a generally hemispherical shape. Dome-shaped wall 90 of eye 20 comprises a cornea 36 and scleral tissue 34. The scleral tissue 34 meets the cornea 36 at a limbus 38 of eye 20. Additional scleral tissue 34 of eye 20 surrounds a posterior chamber PC filled with a viscous fluid known as vitreous humor. A lens 40 of eye 20 is located between anterior chamber AC and posterior chamber PC. Lens 40 is held in place by a number of ciliary zonules 42.

Whenever a person views an object, he or she is viewing that object through the cornea, the aqueous humor, and the lens of the eye. In order to be transparent, the cornea and the lens can include no blood vessels. Accordingly, no blood flows through the cornea and the lens to provide nutrition to these tissues and to remove wastes from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye.

In the illustration of FIG. 6, the cutting plane passing through the center of pupil 32 has also passed through Schlemm's canal. Accordingly, two laterally cut ends of Schlemm's canal SC are visible in the cross-sectional view of FIG. 6. In a healthy eye, aqueous humor flows out of anterior chamber AC and into Schlemm's canal SC. Aqueous humor exits Schlemm's canal SC and flows into a number of collector channels. After leaving Schlemm's canal SC, aqueous humor is absorbed into the venous blood stream and carried out of the eye.

FIG. 7 is a stylized perspective view depicting the surface 92 that defines anterior chamber AC of the eye shown in FIG. 6. In FIG. 7, surface 92 is shown having a generally hemispherical shape. FIG. 7 may be used to illustrate some fundamental geometric concepts that will be used below to describe the various ocular implant delivery cannula structures. Geometry is a branch of mathematics concerned with the properties of space and the shape, size, and relative position of objects within that space. In geometry, a sphere is a round object in three-dimensional space. All points on the surface of a sphere are located the same distance r from a center point so that the sphere is completely symmetrical about the center point. In geometry, a point represents an exact location. A point is a zero-dimensional entity (i.e., it has no length, area, or volume). Geometrically speaking, at any point on a spherical surface, one can find a normal direction which is at right angles to the surface. For a spherical surface all normal directions intersect the center point of the sphere. Each normal direction will also be perpendicular to a line that is tangent to the spherical surface. In FIG. 7, a normal line N is illustrated using dashed lines. Normal line N is at right angles to spherical surface 92. Normal line N is also perpendicular to a reference line TAN. Reference line TAN is tangent to spherical surface 92 in FIG. 7.

A method in accordance with this detailed description may include the step of advancing a distal portion of a cannula into the anterior chamber of the eye. The cannula may then be used to access Schlemm's canal, for example, by piercing the wall of Schlemm's canal with the distal end of the cannula. An ocular implant may be advanced out of the distal opening of the cannula and into Schlemm's canal. A path 94 taken by an ocular implant as it follows Schlemm's canal along surface 92 is illustrated using a row of dots in FIG. 7.

Scleral tissue above the trabecular meshwork, and the scleral spur below the trabecular meshwork, are harder than the meshwork tissue. If the physician advances the cannula's distal tip against the scleral tissue above the canal, the angle of the scleral tissue with respect to the approach angle of the cannula, as well as the hardness of that tissue, will tend to guide the cannula tip downward toward and into the meshwork. This effect can be enhanced if the cannula's distal tip is sharp enough to easily penetrate the meshwork but not sharp enough to easily pierce scleral tissue. If, on the other hand, the physician advances the cannula's distal tip onto the scleral spur below the meshwork, the cannula is likely to miss the meshwork and Schlemm's canal altogether.

Likewise, as the ocular implant advances into Schlemm's canal, the ocular implant may press against the scleral tissue supporting the outer major wall of Schlemm's canal and the scleral tissue of the dome-shaped wall that defines the anterior chamber of the eye. As the body of the ocular implant presses against the dome-shaped wall of the eye, the dome-shaped wall provides support for Schlemm's canal and the ocular implant. The support provided by the dome-shaped wall may be represented by force vectors. The direction of these force vectors may be at right angles to points on the spherical surface that defines the anterior chamber. Accordingly, the outer major wall of Schlemm's canal may be supported by the dome shaped wall as the ocular implant advances circumferentially into Schlemm's canal.

During delivery, it is desirable that the ocular implant follow the lumen of Schlemm's canal as it is advanced out the distal opening of the cannula. The ability of the ocular implant to be advanced into and follow the lumen of Schlemm's canal may be referred to as trackability. Characteristics of an ocular implant that effect trackability include axial pushability and lateral flexibility. Axial pushability generally concerns the ability of an ocular implant to transmit to the distal end of the ocular implant an axial force applied to the proximal end of the ocular implant. Lateral flexibility concerns the ease with which the ocular implant body can bend to conform to the shape of the lumen. Trackability may be adversely affected when twisting forces are applied to a curved body. For example, twisting the body of a curved ocular implant about its longitudinal axis may cause the curved body to steer away from a desired path.

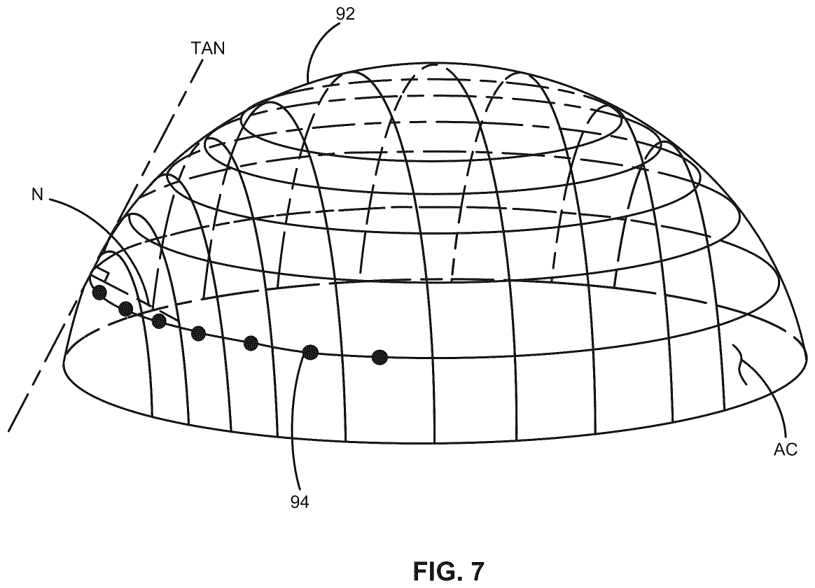

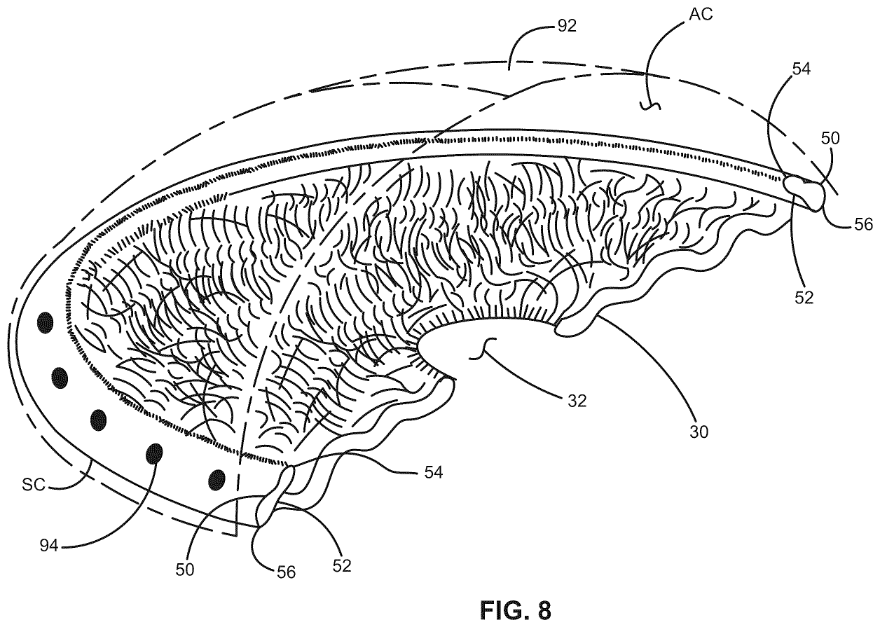

FIG. 8 is a stylized perspective view further illustrating Schlemm's canal SC and iris 30 shown in FIG. 6. The surface 92 that defines the anterior chamber AC of eye 20 is depicted using dashed lines in FIG. 8. In the embodiment of FIG. 8, Schlemm's canal SC and iris 30 are shown in cross-section, with a cutting plane passing through the center of a pupil 32 defined by iris 30. Schlemm's canal SC comprises a first major side 50, a second major side 52, a first minor side 54, and a second minor side 56. Schlemm's canal SC forms a ring around iris 30 with pupil 32 disposed in the center of that ring. With reference to FIG. 8, it will be appreciated that first major side 50 is on the outside of the ring formed by Schlemm's canal SC and second major side 52 is on the inside of the ring formed by Schlemm's canal SC. Accordingly, first major side 50 may be referred to as an outer major side of Schlemm's canal SC and second major side 52 may be referred to as an inner major side of Schlemm's canal SC. With reference to FIG. 8, it will be appreciated that first major side 50 is further from pupil 32 than second major side 52.

A path 94 taken by an ocular implant as it follows Schlemm's canal along surface 92 is illustrated using a row of dots in FIG. 8. As the ocular implant advances into Schlemm's canal, the ocular implant may press against the outer major wall of Schlemm's canal and the dome-shaped wall that defines the anterior chamber.

Some embodiments include an ocular implant delivery cannula with a distal tip that is offset from the longitudinal center line of the cannula. This arrangement facilitates the intuitive use of anatomical landmarks that can be easy observed using gonioscopic visualization. When the body of the cannula is generally centered on Schlemm's canal, the tip portion of the cannula will pierce the trabecular meshwork and the wall of Schlemm's canal at a point slightly above the center of Schlemm's canal. The offset distal tip also provides the distal end of the cannula with a lower camming surface for guiding the cannula distal end over the scleral spur and an optional upper camming surface for guiding the cannula distal end into Schlemm's canal when the cannula has a diameter larger than a width of Schlemm's canal. The camming surfaces are configured to direct the cannula into Schlemm's canal when the cannula is wider or oversized with respect to a width of the canal.

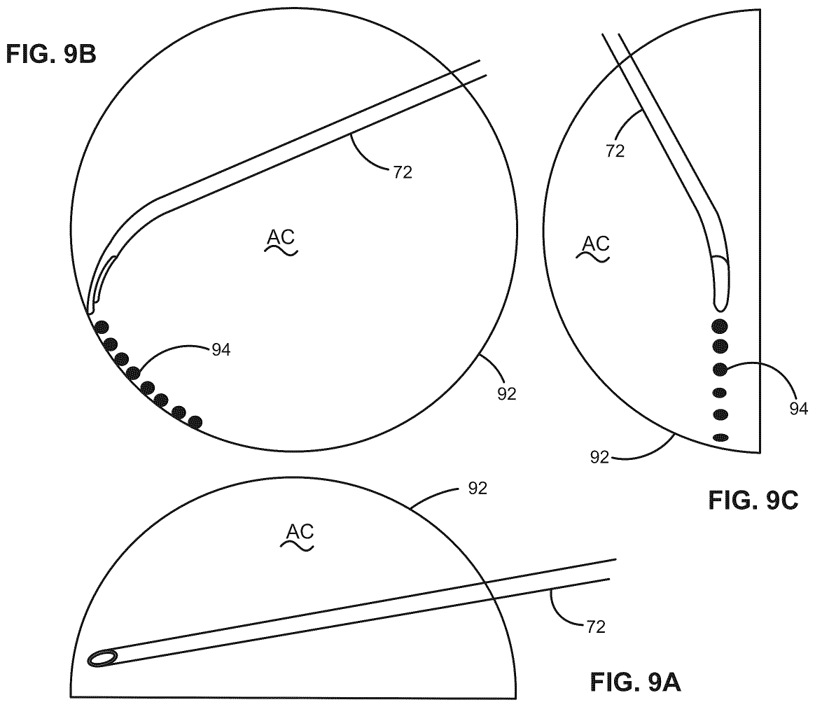

FIGS. 9A-9C are plan views of the surface 92 that defines anterior chamber AC of the eye shown in FIG. 6. FIG. 9A may be referred to as a front view of surface 92, FIG. 9B may be referred to as a top view of surface 92, and FIG. 9C may be referred to as a side view of surface 92.

In FIGS. 9A-9C, a cannula 72 is shown extending into anterior chamber AC. Cannula 72 may be used to deliver an ocular implant to a target location within anterior chamber AC. Examples of target locations that may be suitable in some applications include areas in and around Schlemm's canal, the trabecular meshwork, and the suprachoroidal space of an eye. A path 94 that may be taken by an ocular implant as it follows Schlemm's canal along surface 92 is illustrated using a row of dots in FIGS. 9A-9C.

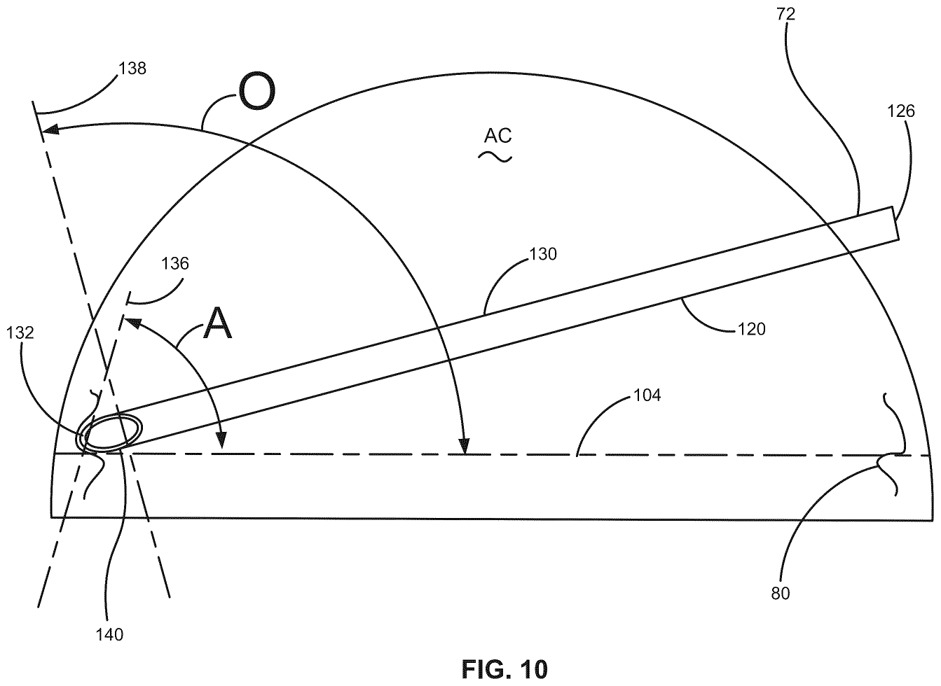

FIG. 10 is an enlarged side view showing cannula 72 extending into anterior chamber AC defined by surface 92. Cannula 72 may be used, for example, to deliver an ocular implant to a target location within Schlemm canal SC. In the stylized plan view of FIG. 10, a scleral spur 80 is disposed in anterior chamber AC. Scleral spur 80 is fixed to surface 92 and encircles anterior chamber AC. Scleral spur 80 defines a spur plane 104.

Referring still to FIG. 10, cannula 72 can include a body member 120 extending along a longitudinal axis. Body member 120 can include a proximal end 126 and a tubular portion 130 extending distally from the proximal end. Body member 120 can also include a tip portion 132 disposed at a distal end thereof. A trough portion 140 of body member extends between tip portion 132 and tubular portion 130. In the embodiment of FIG. 10, tip portion 132 has a semi-circular transverse cross-section including a tip chord line segment. A secant 136 extending beyond the tip chord is shown in FIG. 10. Trough portion 140 of body member 120 has a semi-circular transverse cross-section including a trough chord line segment. FIG. 10 includes a secant 138 extending beyond the trough cord.

As shown in FIG. 10, tip portion 132 and trough portion 140 are adapted and configured such that, when tubular portion 130 is extending through an incision in the dome shaped wall defining anterior chamber AC and tip portion 132 is extending into Schlemm's canal of the eye, secant 136 intersects spur plane 104 at an acute angle A and secant 138 intersects spur plane 104 at an obtuse angle 0.

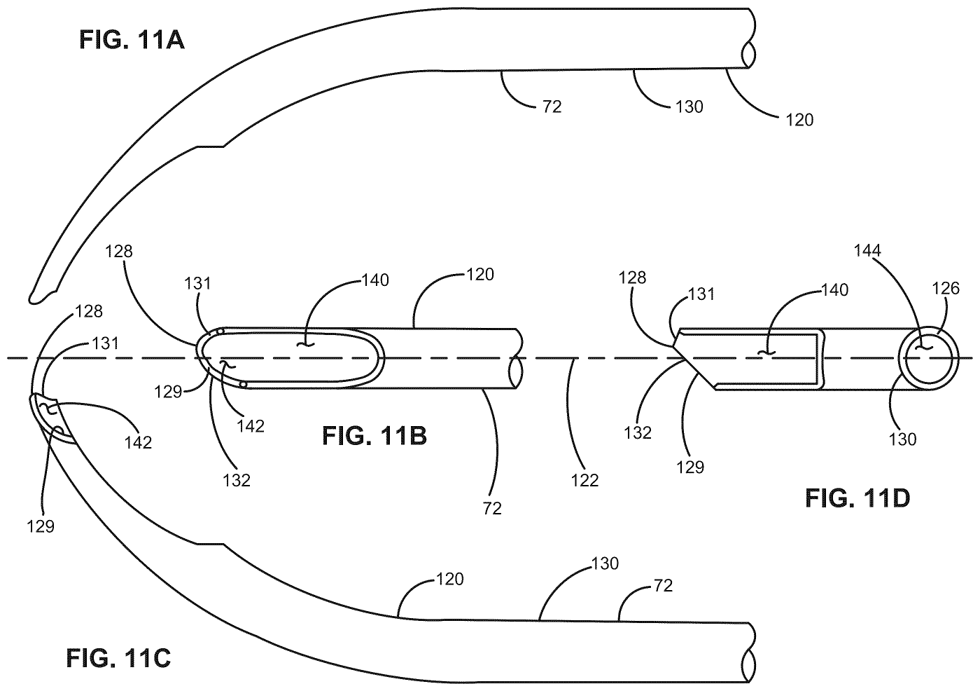

FIGS. 11A-11C are plan views of cannula 72 created using multiview projection. FIG. 11D is an axial view further illustrating cannula 72. Cannula 72 of FIGS. 11A-11D may be used to deliver an ocular implant into Schlemm's canal of an eye. FIG. 11A may be referred to as a top view of cannula 72, FIG. 11B may be referred to as a side view of cannula 72, and FIG. 11C may be referred to as a bottom view of cannula 72.

In FIGS. 11A-11D, cannula 72 comprises a body member 120 extending along a medial plane 122. Body member 120 can include a proximal end 126 and a tubular portion 130 extending distally from the proximal end. Body member 120 can also include a tip portion 132 disposed at a distal tip 128 thereof. The distal tip 128 can be offset from the medial plane 122 of body member 120. The distal tip 128 can form a point at the intersection of lower camming surface 129 and upper camming surface 131. In one alternative embodiment, the distal tip may be at one side of the cannula, in which case there will be no upper camming surface. In some embodiments, distal tip 128 can be sharpened enough to pierce trabecular meshwork tissue but not sharp enough to easily pierce scleral tissue.

Body member 120 also includes a trough portion 140 extending between distal tip 128 and tubular portion 130. Trough portion 140 is configured to fluidly communicates with a lumen 144 defined by tubular portion 130 and a distal opening 142 defined by tip portion 132. Because of the offset position of distal tip 128, tip portion 132 is asymmetric about medial plane 122 and trough portion 140 is symmetric about medial plane 124.

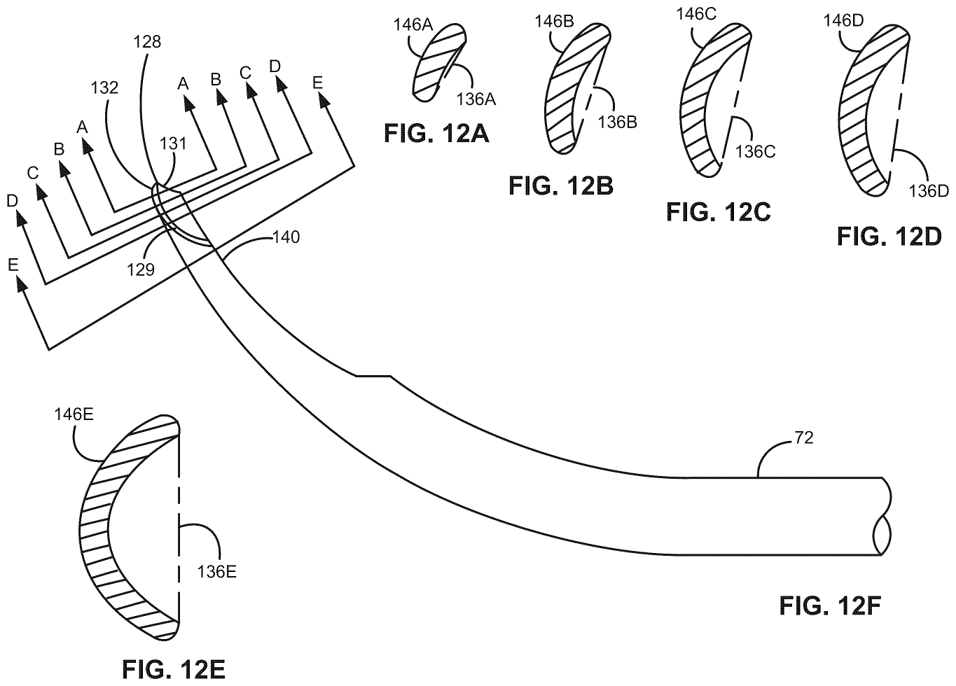

FIG. 12A through FIG. 12D are lateral cross-sectional views of tip portion 132 of cannula 72. FIG. 12E is a lateral cross-sectional view of trough portion 140 of cannula 72. FIG. 12F is an enlarged plan view showing a portion of cannula 72 shown in the previous figure. In this embodiment, the cannula is formed from a tube (such as a hypotube) with material removed from the distal end to form the trough portion and the distal tip 129. In other embodiments, the cannula may have a non-tubular shape. FIG. 12F shows the cannula 72 including the tip portion 132, distal tip 128, camming surfaces 129 and 131, and trough portion 140. In FIG. 12F, a number of section lines can be seen traversing crossing cannula 72. These section lines have been used to create a number of lateral cross-sections illustrating the shape of cannula 72.

Section 146A of FIG. 12A was created by cutting tip portion 132 along section line A-A shown in FIG. 12F. Section 146B, section 146C, and section 146D, of FIGS. 12B, 12C, and 12D, respectively, were made by cutting tip portion 132 along section line B-B, section line C-C, and section line D-D, respectively. By examining section 146A, section 146B, section 146C and section 146D it will be appreciated that tip portion 132 can have a semi-circular transverse cross-section.

As shown in FIGS. 12A-12D, section 146A has a chord 136A. Section 146B, section 146C, and section 146D have a chord 136B, a chord 136C and a chord 136D, respectively. By examining chord 136A, chord 136B, chord 136C and chord 136D it will be appreciated that the chord length of tip portion 132 increases as tip portion 132 extends proximally away from its distal point. Section 146E was created by cutting through portion 140 along section line E-E shown in FIG. 12F. In the embodiment of FIG. 12E, section 146E has a chord 136E.

Referring to FIGS. 11A-11D and 12A-12E, as the physician advances the cannula through the anterior chamber toward the trabecular meshwork under visual guidance (using, e.g., the scleral spur, pigmented area and Schwalbe's line as anatomical landmarks), the camming surfaces 129 and 131 and the cannula's tip portion 132 are configured to guide an oversized cannula relative to the width of Schlemm's canal into Schlemm's canal. In some embodiments, a diameter of the cannula can be between approximately 350-550 microns, or alternatively, between 400-500 microns. Schlemm's canal typically has a width of approximately 300 microns, so it can be a challenge to guide a conventional cannula that is wider than Schlemm's canal into the canal. In the present embodiment, the upper camming surface 131 of the cannula will engage scleral tissue 9 above the meshwork. Since the distal tip 128 is not sharp enough to easily pierce scleral tissue, upper camming surface 131 is configured to contact the scleral tissue and guide the distal tip 128 into Schlemm's canal. The lower camming surface 129 is configured to contact the scleral spur below the meshwork to guide the tip 128 into the Schlemm's canal. The distal tip's offset, placing it above the cannula's longitudinal center axis, along with the physician's use of the anatomical landmarks, helps ensure that the cannula is not positioned so low with respect to the meshwork that the upper camming surface engages the scleral spur to push the cannula tip downward away from the meshwork.

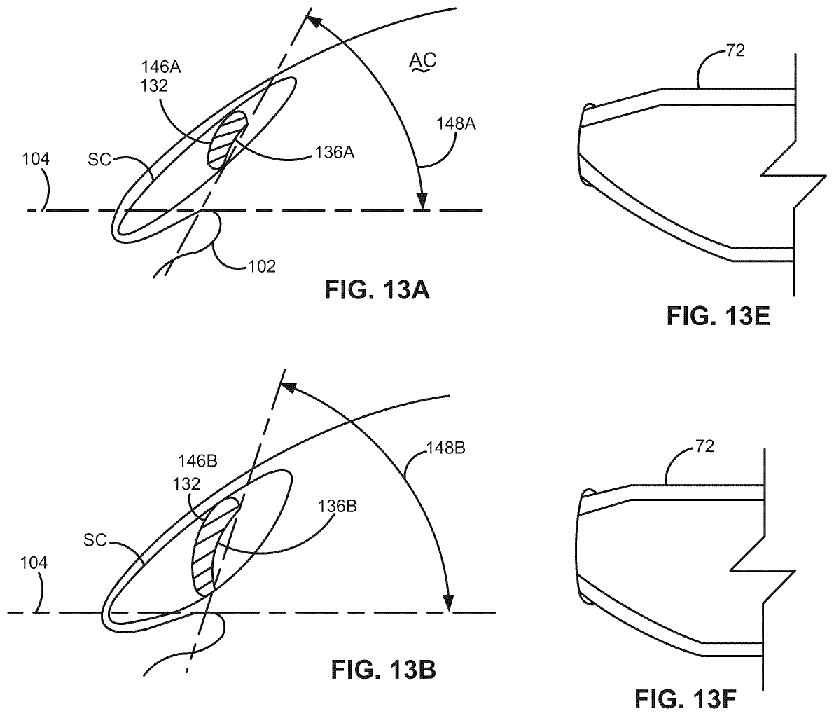

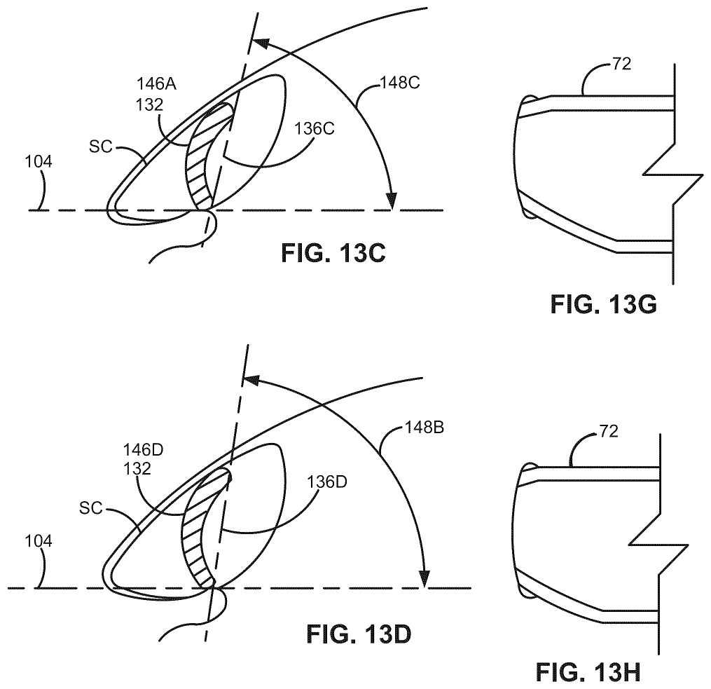

FIGS. 13A-13D form a sequence of stylized section views illustrating the insertion of tip portion 132 of cannula 72 into Schlemm's canal SC located in the anterior chamber AC of an eye. FIGS. 13E-13H form a sequence stylized side plan views further illustrating the insertion of the tip portion into Schlemm's canal.

In FIG. 13A and FIG. 13E, tip portion 132 of cannula 72 has been advanced into Schlemm's canal so that section 146A (shown in FIG. 12A) of tip portion 132 is aligned with the incision in Schlemm's canal created by the cannula's distal tip 128. Section 146A includes a chord 136A. Referring to FIG. 13A, chord 136A defines a line that intersects a spur plane 104 of the eye at a chord angle 148A. Spur plane 104 is defined by a scleral spur 102 that encircles the anterior chamber AC of the eye.

In FIG. 13B and FIG. 13F, tip portion 132 of cannula 72 has been advanced into Schlemm's canal so that section 146B of tip portion 132 is aligned with the incision in Schlemm's canal. Section 146B includes a chord 136B. In FIG. 13B, chord 136B defines a line that intersects spur plane 104 at a chord angle 148B.

In FIG. 13C and FIG. 13G, tip portion 132 of cannula 72 has been advanced into Schlemm's canal so that section 146C of tip portion 132 is aligned with the incision in Schlemm's canal. Section 146C includes a chord 136C. In FIG. 13C, chord 136C defines a line that intersects spur plane 104 at a chord angle 148C.

In FIG. 13D and FIG. 13H, tip portion 132 of cannula 72 has been advanced into Schlemm's canal so that section 146D of tip portion 132 is aligned with the incision in Schlemm's canal. Section 146D includes a chord 136D. In the embodiment of FIG. 13D, chord 136D defines a line that intersects spur plane 104 at a chord angle 148D.

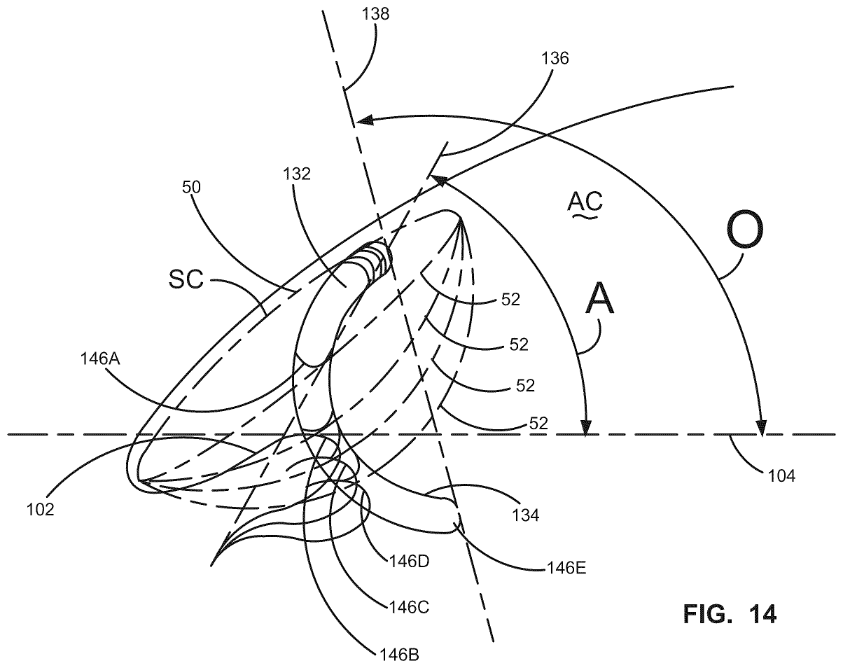

FIG. 14 is an abstract graphical representation further illustrating the insertion of tip portion 132 of a cannula into Schlemm's canal SC. The profile of each section view illustrated in FIGS. 12A-12D is included in FIG. 14. These profiles form contour lines illustrating the tapered shape of tip portion 132 and trough portion 140. The profiles associated with section 146A, section 146B, section 146C, section 146D, and section 146E are labeled in FIG. 14.

As tip portion 132 is inserted into Schlemm's canal, inner major wall 52 of Schlemm's canal rides along a first leading edge of tip portion 132. The insertion of tip portion 132 into Schlemm's canal SC causes inner major wall 52 to separate from outer major wall 50. The changing shape of Schlemm's canal is illustrated with a plurality of Schlemm's canal profiles shown using dashed lines in FIG. 14.

In the embodiment of FIG. 14, tip portion 132 and trough portion 140 are adapted and configured such that, when tubular portion 130 is extending through an incision in the dome shaped wall defining anterior chamber AC and tip portion 132 is extending into Schlemm's canal of the eye, secant 136 intersects spur plane 104 at an acute angle A and secant 138 intersects spur plane 104 at an obtuse angle 0.

While embodiments of the present invention have been shown and described, modifications may be made, and it is therefore intended in the appended claims to cover all such changes and modifications which fall within the true spirit and scope of the invention.

What is claimed is:

1. A cannula for delivering an ocular implant into Schlemm's canal of an eye, comprising:
a curved tube comprising a lumen configured to direct the ocular implant to a location within Schlemm's canal;
a trough portion disposed distally of the curved tube; and
a distal portion disposed distally of the trough portion having a distal tip, wherein the distal tip is offset with respect to a medial plane of the curved tube.

2. The cannula of claim 1, wherein the distal tip is formed by a point at an intersection of a lower camming surface and an upper camming surface.

3. The cannula of claim 2, wherein the upper camming surface is shorter than the lower camming surface.

4. The cannula of claim 2, wherein the lower camming surface is configured to guide the cannula over a scleral spur of the eye.

5. The cannula of claim 4, wherein the distal tip is blunt such that the distal tip does not pierce scleral tissue.

6. The cannula of claim 1, wherein the distal tip is offset from a central axis of the trough portion.

7. The cannula of claim 1, wherein the distal portion is asymmetric with respect to the medial plane of the curved tube.

8. The cannula of claim 1, wherein the distal portion is defined by an asymmetric V-shape.

9. The cannula of claim 1, wherein the trough portion is symmetric with respect to the medial plane.

10. The cannula of claim 1, where the cannula is shaped such that at least part some of the trough portion is advanced into Schlemm's canal while a first portion of the curved tube is disposed inside an anterior chamber of the eye and a second portion of the curved tube is extended through an incision in the eye to a location outside of the eye.

11. An ophthalmic system, comprising:
a cannula comprising:
a curved tube comprising a lumen,
a trough portion disposed distally of the curved tube, and
a distal portion disposed distally of the trough portion, wherein the distal portion is defined by an asymmetric V-shape; and
an ocular implant configured to be advanced distally through the lumen and along the trough portion into Schlemm's canal of an eye.

12. The system of claim 11, wherein the distal portion is formed by a lower camming surface and an upper camming surface.

13. The system of claim 12, wherein the distal portion is asymmetric with respect to a medial plane of the curved tube due to the upper camming surface being shorter than the lower camming surface.

14. The system of claim 12, wherein the lower camming surface is configured to guide the cannula over a scleral spur of the eye.

15. The system of claim 11, wherein the distal portion is asymmetric with respect to a medial plane of the curved tube.

16. The system of claim 11, wherein the distal portion comprises a blunt distal tip.

17. A method for delivering an ocular implant into Schlemm's canal of an eye, comprising:
advancing a cannula into the eye, wherein the cannula comprises a curved tube, a trough portion disposed distally of the curved tube, and a distal portion disposed distally of the trough portion, wherein the distal portion comprises a distal tip, wherein the distal tip is offset with respect to a medial plane of the curved tube;
inserting the distal portion into Schlemm's canal such that at least part of the trough portion is in fluid communication with Schlemm's canal; and
advancing the ocular implant through a lumen of the curved tube and into Schlemm's canal.

18. The method of claim 17, further comprising allowing a lower camming surface of the distal portion to contact a scleral spur of the eye to guide the distal portion and at least part of the trough portion into Schlemm's canal.

19. The method of claim 18, further comprising allowing an upper camming surface of the distal portion to contact scleral tissue of the eye to guide the distal portion and at least part of the trough portion into Schlemm's canal.

20. The method of claim 17, wherein the distal portion is defined by an asymmetric V-shape.
